(19) United States
(12) Patent Application Publication
(10) Pub. No.: US 2022/0334064 A1
(43) Pub. Date: Oct. 20, 2022

(54) METHODS, APPARATUSES, AND SYSTEMS FOR PROVIDING GAS-SENSITIVE SUBSTRATES

(71) Applicant: Honeywell International, Inc., Charlotte, NC (US)

(72) Inventors: Shrinidhi Dawande, Arlington Heights, IL (US); Jimmy Shin, Lake Zurich, IL (US); Jingnian Zhang, Palatine, IL (US); Tamami Yamaguchi, Lake Forest, IL (US)

(21) Appl. No.: 17/301,919

(22) Filed: Apr. 19, 2021

(51) Int. Cl. G01N 21/78; G01N 33/00

(52) U.S. Cl. CPC G01N 21/783; G01N 33/0063

(57) ABSTRACT

Methods, apparatuses and systems for providing gas-sensitive substrates are disclosed herein. An example apparatus may comprise: a gas-sensitive substrate at least partially disposed within a gas flow channel of the gas detecting apparatus, wherein at least a portion of the gas-sensitive substrate is treated to mimic stain characteristics produced by at least one interferent gaseous substance, and the gas-sensitive substrate is configured to produce a stain in response to making contact with at least one target gaseous substance disposed within the gas flow channel.

### BACKGROUND

Gas detecting apparatuses may comprise gas-sensitive substrates which may be utilized to detect and/or measure the concentration level of gaseous substances and/or compounds in a gaseous substance, including, for example, organic compounds and inorganic compounds. Many gas detecting apparatuses and gas-sensitive substrates are plagued by technical challenges and limitations.

### BRIEF SUMMARY

Various embodiments described herein relate to methods, apparatuses, and systems for providing gas-sensitive substrates.

In accordance with various examples of the present disclosure, an apparatus is provided.

In some examples, the apparatus comprises a gas-sensitive substrate at least partially disposed within a gas flow channel of the gas detecting apparatus, wherein at least a portion of the gas-sensitive substrate is treated to mimic stain characteristics produced by at least one interferent gaseous substance, and the gas-sensitive substrate is configured to produce a stain in response to making contact with at least one target gaseous substance disposed within the gas flow channel.

In accordance with various examples of the present disclosure, a method for manufacturing a gas-sensitive substrate configured to produce a stain in response to making contact with at least one target gaseous substance is provided. In some examples, the method comprises treating at least a portion of a substrate to mimic stain characteristics produced by at least one interferent gas and depositing at least one gas-sensitive material unto the substrate.

The foregoing illustrative summary, as well as other exemplary objectives and/or advantages of the disclosure, and the manner in which the same are accomplished, are further explained in the following detailed description and its accompanying drawings.

### BRIEF DESCRIPTION OF THE DRAWINGS

The description of the illustrative embodiments may be read in conjunction with the accompanying figures. It will be appreciated that, for simplicity and clarity of illustration, elements illustrated in the figures have not necessarily been drawn to scale, unless described otherwise. For example, the dimensions of some of the elements may be exaggerated relative to other elements, unless described otherwise. Embodiments incorporating teachings of the present disclosure are shown and described with respect to the figures presented herein, in which:

FIG. 1 illustrates an example schematic diagram depicting a portion of an example apparatus in accordance with various embodiments of the present disclosure;

FIGS. 2A and 2B illustrate example schematic diagrams depicting gas-sensitive substrates in accordance with various embodiments of the present disclosure;

FIG. 3 is a flowchart diagram illustrating an example method in accordance with various embodiments of the present disclosure;

FIG. 4 illustrates an example controller component in electronic communication with other component(s) of an example gas detecting apparatus in accordance with various embodiments of the present disclosure; and

FIG. 5 is a flowchart diagram illustrating example operations in accordance with various embodiments of the present disclosure.

### DETAILED DESCRIPTION OF THE INVENTION

Some embodiments of the present disclosure will now be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

The components illustrated in the figures represent components that may or may not be present in various embodiments of the present disclosure described herein such that embodiments may include fewer or more components than those shown in the figures while not departing from the scope of the present disclosure. Some components may be omitted from one or more figures or shown in dashed line for visibility of the underlying components.

The phrases “in an example embodiment,” “some embodiments,” “various embodiments,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such components or features may be optionally included in some embodiments, or may be excluded.

The term “electronically coupled” or “in electronic communication with” in the present disclosure refer to two or more electrical elements (for example, but not limited to, an example processing circuitry, communication module, input/output module memory, humidity sensing component, cooling element, gas detection component) and/or electric circuit(s) being connected through wired means (for example but not limited to, conductive wires or traces) and/or wireless means (for example but not limited to, wireless network, electromagnetic field), such that data and/or information (for example, electronic indications, signals) may be transmitted to and/or received from the electrical elements and/or electric circuit(s) that are electronically coupled.

Various apparatuses (such as, but not limited to, a gas detecting apparatus) may detect and/or measure the concentration level of a target gaseous substance or family of target gaseous substances, in some examples, within a specified location or area. Target gaseous substances may include volatile organic compounds (VOCs), toxic gases and the like. In some examples, gas-sensitive substrates (e.g., chemically impregnated paper tapes) comprising chemicals/materials sensitive to one or more particular target gaseous substances may be used in conjunction with a gas detecting apparatus. For example, a sample gaseous substance may be directed to flow through an example gas detecting apparatus such that it makes contact with (e.g., passes through) an example gas-sensitive substrate. If a particular target gaseous substance is present in the sample gaseous substance, the example gas-sensitive substrate sensitive to that target gaseous substance may undergo a chemical reaction that may produce a stain on the gas-sensitive substrate (i.e., cause the gas-sensitive substrate to change color). In some examples, the stain characteristics (e.g., stain density or color change) may be calibrated to correspond to a concentration of the specified target gaseous substance or family of target gaseous substances. In various examples, optical components/systems may be used to detect and measure characteristics of the stain (e.g., stain density, color change and/or color intensity of a stain developing over time) on the gas-sensitive substrate in order to determine the concentration of the target gaseous substance(s) in the sample gaseous substance.

Many gas detecting apparatuses and gas-sensitive substrates for detecting the presence of one or more target gaseous substances as described above may present many technical challenges and limitations.

In some examples, a gas-sensitive substrate may be treated or formulated to react with a family of target gaseous substances such that it undergoes a chemical reaction thereby producing a stain with particular characteristics (e.g., particular stain density and/or color change) in response to making contact with any one of a particular family of target gaseous substances. An example family of target gaseous substances may be or comprise Hydrides, Mineral Acids, Oxidizers, Amines, Diisocyanates, Hydrazines or the like. However, in some examples, a gas-sensitive substrate may be treated or formulated to react with a particular target gaseous substance. In various examples, interferent gas(es) having similar chemical properties and/or inducing similar reactivities may produce stains in similar ways and with similar characteristics such that an optical system cannot accurately differentiate between a target gaseous substance and an interferent gaseous substance (such as, but not limited to, an interferent gaseous substance within the same family as the target gaseous substance). By way of example, a gas-sensitive substrate treated to react with Fluoride may undergo a similar reactivity and thus exhibit similar stain characteristics (e.g., stain density and/or color change) in response to Chlorine. As such, a gas detecting apparatus may be unable to accurately determine whether a sample gaseous substance comprises Fluoride or Chlorine. This may result in inaccurate readings and alarms being generated by the example gas detecting apparatus in some applications.

In accordance with various embodiments of the present disclosure, example methods, apparatuses and systems are provided.

For example, the present disclosure may provide a gas detecting apparatus comprising a gas-sensitive substrate at least partially disposed within a gas flow channel of the gas detecting apparatus. At least a portion of the gas-sensitive substrate may be treated to mimic stain characteristics produced by at least one interferent gaseous substance. The gas-sensitive substrate may be configured to produce a stain in response to making contact with at least one target gaseous substance disposed within the gas flow channel. The example gas detecting apparatus may comprise an optical component configured to generate a concentration level indication associated with the at least one target gaseous substance in the gas flow channel. The optical component may comprise a gas detecting element. The example gas detecting apparatus may comprise a controller component in electronic communication with the gas detecting element. The controller component may be configured to receive the concentration level indication from the optical component, determine whether the concentration level indication satisfies a concentration level threshold, and in response to determining that the concentration level indication satisfies the concentration level threshold, generate a warning indication. The at least one target gaseous substance may comprise an Oxidizer. The at least one target gaseous substance may comprise one or more of Chlorine, Fluorine, Nitrogen Dioxide and Chlorine Dioxide. In some examples, a color of the gas-sensitive substrate may be yellow. In some examples, a color of the gas-sensitive substrate may be grey. The at least one interferent gaseous substance may comprise one or more of Ozone, Sulphur Dioxide and Hydrogen Peroxide. The gas-sensitive substrate may treated with an alcohol-based or solvent-based solution. In some examples, the gas-sensitive substrate may be heated or dried.

In some examples, a method for manufacturing a gas-sensitive substrate configured to produce a stain in response to making contact with at least one target gaseous substance is provided. The method may comprise treating at least a portion of a substrate to mimic stain characteristics produced by at least one interferent gas, and depositing at least one gas-sensitive material unto the substrate. In some examples, the at least one target gaseous substance may comprise an Oxidizer. The at least one target gaseous substance may comprise one or more of Chlorine, Fluorine, Nitrogen Dioxide and Chlorine Dioxide. In some examples, a color of the gas-sensitive substrate may be yellow. In some examples, a color of the gas-sensitive substrate may be grey. The at least one interferent gaseous substance may comprise one or more of Ozone, Sulphur Dioxide and Hydrogen Peroxide. The gas-sensitive substrate may comprise a paper tape. The method may comprise treating the substrate with an alcohol-based or solvent-based solution. The method may comprise heating or drying the substrate.

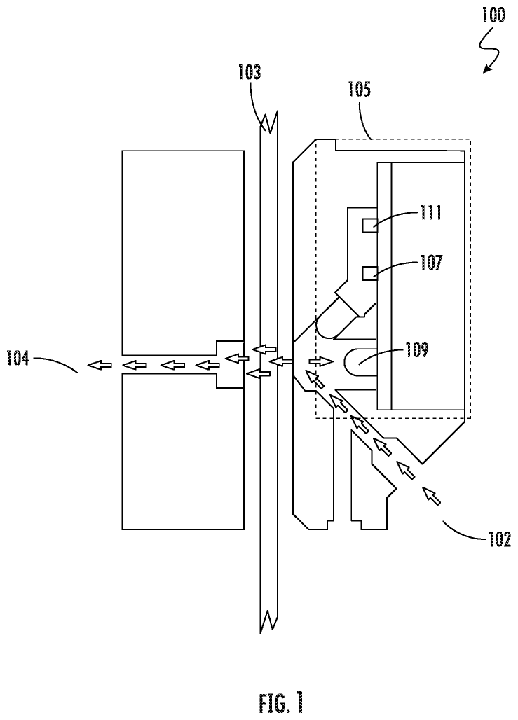

Referring now to FIG. 1, an example schematic diagram depicting a portion of a gas detecting apparatus 100 in accordance with some embodiments of the present disclosure is provided. In particular, as shown, the gas detecting apparatus 100 comprises a gas-sensitive substrate 103 and an optical component 105.

In particular, the example gas detecting apparatus 100 may be configured to detect and/or measure a concentration of a target gaseous substance in a sample gaseous substance (e.g., air sample) within a gas flow channel of the gas detecting apparatus 100. The target gaseous substance may be or comprise, for example but not limited to, a Hydride, a Mineral Acid, an Oxidizer, an Amine, a Diisocyanate or a Hydrazine. In various examples, the gas detecting apparatus 100 may be configured to detect and/or measure a concentration of a particular gaseous substance or a particular family of gaseous substances. By way of example, the example gas detecting apparatus 100 may be configured to detect a particular Oxidizer (e.g., Chlorine, Fluorine, Nitrogen Dioxide or the like).

As depicted in FIG. 1, a sample gaseous substance (such as an air sample) may flow through at least a portion of the example gas detecting apparatus 100 such that the sample gaseous substance (e.g., air sample) is incident on/makes contact with the gas-sensitive substrate 103 resulting in a chemical reaction. For example, as depicted in FIG. 1, the gas detecting apparatus 100 may be configured to receive a sample gaseous substance (e.g., air sample) and convey the gaseous substance from a gas inlet 102 through a gas flow channel to be expelled from the gas detecting apparatus (e.g., caused to exit the apparatus) via a gas outlet 104.

As depicted, the gas detecting apparatus 100 comprises a gas flow channel. As shown, the gas flow channel may refer to a passageway beginning with the gas inlet 102 and terminating at the gas outlet 104 through which a gaseous substance (such as, but not limited to, an air sample) may enter, flow through and be expelled from the gas detecting apparatus 100. The gas flow channel may be or comprise, for example, without limitation, a pipe, conduit, tubular structure and/or the like.

Referring again to FIG. 1, a gaseous substance (for example, an air sample) may enter the gas detecting apparatus 100 through the gas inlet 102. In some embodiments, the gas inlet 102 may refer to an opening on a surface of a housing of the gas detecting apparatus 100. Similarly, in some embodiments, the gas outlet 104 may refer to an opening on a surface of the housing of the gas detecting apparatus 100 that may be distinct from the opening on the surface associated with the gas inlet 102. The gas inlet 102 and the gas outlet 104 may be connected by the gas flow channel and define the beginning point and end point of the gas flow channel, respectively. In some embodiments, the gaseous substance may be conveyed in a direction due to air flow generated by a pump. For example, the pump may generate air flow in the gas flow channel such as by pulling in (e.g., drawing in) a gaseous substance (e.g., input air) through the gas inlet 102 and pushing the gaseous substance (e.g., output air) through the gas outlet 104. Thus, the gaseous substance is received through an opening (e.g., gas inlet 102) leading to the gas flow channel and conveyed to another opening (e.g., gas outlet 104) where it may be expelled from the gas detecting apparatus 100. In some embodiments, the pump may be or comprise, for example, without limitation a compressor, a vacuum pump, a manual pump, a motorized pump or the like.

As noted above, and as depicted in FIG. 1, the gas detecting apparatus 100 comprises a gas-sensitive substrate 103. As shown, the gas-sensitive substrate 103 is at least partially disposed within the flow channel of the gas detecting apparatus 100 such that a sample gaseous substance will make contact with (i.e., pass through) or otherwise interact with at least a portion of the gas-sensitive substrate 103 as it travels from the gas inlet 102 of the gas detecting apparatus 100, through the gas flow channel and to the gas outlet 104 of the gas detecting apparatus 100. In some examples, the gas-sensitive substrate 103 may be or comprise a paper-based or plastic-based material/media capable of receiving (e.g., being impregnated with) gas-sensitive material(s) such as, in some examples, chemical reagents. The example chemical reagent may operate to provide a detectable indication (e.g., a visual indication) that one or more particular gaseous substances or family of gaseous substances is present in a sample gaseous substance. In some examples, the gas-sensitive substrate 103 may comprise a tape, cartridge, cassette or the like which may be wound on a reel so that it can be dispensed within the gas detecting apparatus 100. For example, an example gas-sensitive substrate (e.g., paper-based, plastic-based tape, combinations thereof, and/or the like) may be dispensed within the gas detecting apparatus 100 incrementally over time such that a different (e.g., unused) portion of the gas-sensitive substrate is utilized to periodically take measurements of sample gaseous substance(s) within a gas flow channel of the gas detecting apparatus 100. In response to making contact with at least one target gaseous substance, a chemical reaction may occur in the gas-sensitive substrate 103. For example, the gas-sensitive substrate 103 may be configured to produce a stain with certain characteristics (e.g., stain density, color and/or the like) in response to making contact with at least one target gaseous substance disposed within/flowing through the gas flow channel of the gas detecting apparatus 100. In various examples, the target gaseous substance(s) may react with the gas-sensitive substrate 103 and produce a stain with a density that is proportional to a concentration of the target gaseous substance(s).

As noted above, and as further depicted in FIG. 1, the gas detecting apparatus 100 comprises an optical component 105. The optical component 105 may be configured to detect and/or measure a concentration of at least one gaseous substance in the gas flow channel of the gas detecting apparatus 100. In various examples, the optical component 105 may be configured to generate a concentration level indication associated with at least one target gaseous substance in the gas flow channel of the gas detecting apparatus 100.

As depicted in FIG. 1, the optical component 105 comprises at least one light source 107 and at least one gas detecting element 109. In various examples, the at least one light source 107 is configured to illuminate a stain produced by the gas-sensitive substrate 103 resulting from contact with target gaseous substance(s). The at least one light source 107 may be or comprise, for example, without limitation, a light emitting diode (LED), an infrared light source or the like. As depicted, the at least one light source 107 is disposed adjacent the flow path of the gas detecting apparatus 100 so that light generated therefrom is incident on at least a portion of the gas-sensitive substrate 103 disposed within the gas flow channel of the gas detecting apparatus 100.

As noted above, the optical component 105 comprises at least one gas detecting element 109. In various examples, the gas detecting element 109 is configured to optically measure characteristics of a stain (e.g., stain density) illuminated by the at least one light source 107. In some examples, concentration of the target gaseous substance(s) may be measured in parts-per-million (ppm), parts-per-billion (ppb) or milligrams-per-cubic-meter (mg/m3). The example gas detecting element 109 may be or comprise a sensor such as a photodiode, a spectrophotometer, an RGB color sensor, a CCD color image sensor or the like.

In some examples, as further depicted in FIG. 1, the optical component 105 comprises a reference detector 111 which may monitor and/or control the intensity of the light source 107. The optical component 105 may be electronically coupled with a controller component (e.g., a microprocessor) configured to interpret characteristics of the detected stain (e.g., stain density, color change or the like). For example, the controller component may calculate and store a detected concentration level in memory.

While FIG. 1 provides an example of a gas detecting apparatus 100, it is noted that the scope of the present disclosure is not limited to the example shown in FIG. 1. In some examples, an example gas detecting apparatus 100 may comprise one or more additional and/or alternative elements, and/or may be structured/positioned differently than those illustrated in FIG. 1. For example, the gas detecting apparatus 100 in accordance with the present disclosure may comprise more than one optical component 105.

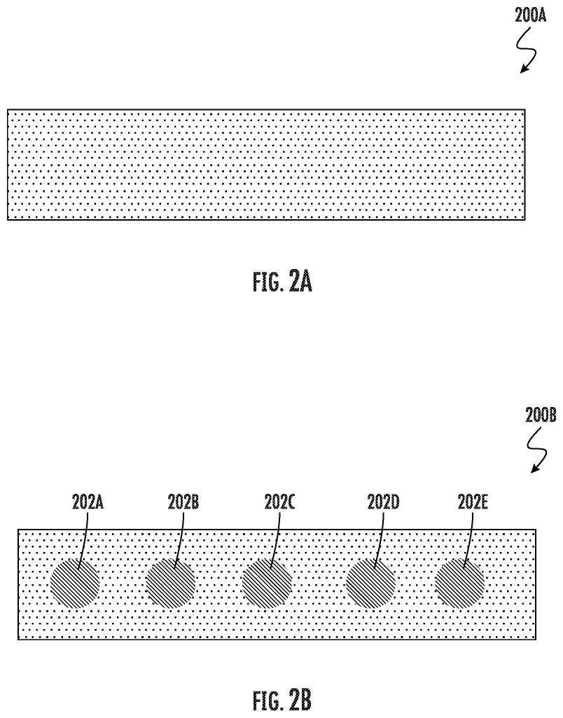

Referring now to FIG. 2A, an example schematic diagram depicting a top view of a gas-sensitive substrate 200A in accordance with various embodiments of the present disclosure is provided. In various examples, at least a portion of the gas-sensitive substrate 200A may be treated to mimic stain characteristics (e.g., stain density, color or the like) produced by at least one interferent gaseous substance. Additionally, the gas-sensitive substrate 200A may configured to produce a stain in response to making contact with at least one target gaseous substance (e.g., disposed within a gas flow channel of an example gas detecting apparatus). By way of example, the gas-sensitive substrate 200A may be configured to detect and/or measure a concentration of a target gaseous substance, Nitrogen Dioxide and not respond to an interferent gaseous substance, Ozone. In the above example, the gas-sensitive substrate 200A may be treated (e.g., chemically treated, dyed or the like) to appear similar to the color of a stain produced by an interferent gaseous substance. For example, if Ozone produces a yellow stain in response to contact with the example gas-sensitive substrate 200A, then the gas-sensitive substrate 200A may be treated to appear yellow. Therefore, in the above example, the gas-sensitive substrate 200A will more accurately detect and measure the concentration of the target gaseous substance, Nitrogen Dioxide, and not respond to the interferent gaseous substance, Ozone.

As depicted in FIG. 2A, the example gas-sensitive substrate 200A comprises a substantially flat, planar, rectangular media. In some examples, the gas-sensitive substrate 200A may be or comprise a paper-based and/or plastic-based tape wound on a reel from which it can be dispensed within an example gas detecting apparatus. In various examples, the gas-sensitive substrate 200A may comprise one or more gas-sensitive regions comprising gas-sensitive material(s) (e.g., chemically impregnated regions). In other examples, as depicted in FIG. 2A, the entire gas-sensitive substrate 200A comprises gas-sensitive material(s). For instance, the entire gas-sensitive substrate 200A may be chemically treated.

In various examples, the gas-sensitive substrate 200A may be configured to detect and/a measure a concentration of different target gaseous substances or different families of target gaseous substances. In some examples, the gas-sensitive substrate 200A may be configured to detect and/or measure a concentration of the same target gaseous substance(s). For example, as the gas-sensitive substrate 200A traverses at least a portion of an example gas-detecting apparatus, measurements may be taken at different respective locations (e.g., points, regions and/or the like) of the gas-sensitive substrate 200A. In some examples, the testing region(s) may be non-continuous or, as depicted in FIG. 2A may cover the entirety of the example gas-sensitive substrate 200A. By way of example, the gas-sensitive substrate 200A may be configured to detect and/or measure a concentration of one or more Oxidizers or one or more Hydrides.

Referring now to FIG. 2B, an example schematic diagram depicting a top view of a gas-sensitive substrate 200B in accordance with various embodiments of the present disclosure is provided. In various examples, at least a portion of the gas-sensitive substrate 200B may be treated to mimic stain characteristics (e.g., stain density, color or the like) produced by at least one interferent gaseous substance. Additionally, the gas-sensitive substrate 200B may configured to produce a stain in response to making contact with at least one target gaseous substance (e.g., disposed within a gas flow channel of an example gas detecting apparatus). By way of example, the gas-sensitive substrate 200B may be configured to detect and/or measure a concentration of a target gaseous substance, Fluoride and not respond to an interferent gaseous substance, Chlorine. In the above example, at least a portion of the gas-sensitive substrate 200B may be treated (e.g., chemically treated, dyed or the like) to appear similar to the color of a stain produced by an interferent gaseous substance. For example, if Chlorine produces a grey stain in response to contact with the example gas-sensitive substrate 200B, then the gas-sensitive substrate 200B may be treated to appear grey. Accordingly, the treated gas-sensitive substrate 200B may remain fully reactive to an example target gaseous substance but will not react to an example interferent gaseous substance (e.g., an interferent gaseous substance that produces stain characteristics that are similar to the treated gas-sensitive substrate 200B). Therefore, in the above example, the gas-sensitive substrate 200B will more accurately detect and measure the concentration of the target gaseous substance, Fluoride, and not react or respond to the interferent gaseous substance, Chlorine. Alternatively, in some examples, only a portion of the example gas-sensitive substrate adjacent a particular gas-sensitive region or testing region may be treated to mimic stain characteristics (e.g., stain density or color) of one or more particular interferent gaseous substances associated with the respective gas-sensitive region or testing region.

As depicted in FIG. 2B, the example gas-sensitive substrate 200B comprises a substantially flat, planar, rectangular media. In some examples, the gas-sensitive substrate 200B may be or comprise a paper-based and/or plastic-based tape wound on a reel from which it can be dispensed within an example gas detecting apparatus. In various examples, the gas-sensitive substrate 200B may comprise one or more gas-sensitive regions comprising gas-sensitive material(s) (e.g., chemically impregnated regions). In other examples, as depicted in FIG. 2B, the entire gas-sensitive substrate 200B comprises gas-sensitive material(s).

As depicted in FIG. 2B, the example gas-sensitive substrate 200B comprises a first testing region 202A, a second testing region 202B, a third testing region 202C, a fourth testing region 202D, and a fifth testing region 202E. As depicted, each testing region 202A, 202B, 202C, 202D and 202E defines a circular area along a surface of the gas-sensitive substrate 200B. In various examples, the testing regions 202A, 202B, 202C, 202D and 202E may continuously extend along the entire length of the gas-sensitive substrate 200B. In some examples, each of the testing regions 202A, 202B, 202C, 202D and 202E may define a location (e.g., point, area, region, or the like) at which an example gas detecting apparatus may draw a sample gaseous substance in order to take measurements. In some examples, more than one testing region may define a testing window such that more than one measurement is generated for an example sample gaseous substance. In various examples, the gas sensitive substrate 200B may be configured to detect the same target gaseous substance(s), different gaseous substance(s), one or more families of gaseous substances, combinations thereof, and/or the like. By way of example, first testing region 202A, the third testing region 202C and the fifth testing region 202E may be configured to detect and/or measure a concentration of Fluoride. In the above example, the second testing region 202B and the fourth testing region 202D may be configured to detect and/or measure a concentration of Nitrogen Dioxide. In some examples, only a portion of the example gas-sensitive substrate 200B adjacent a particular testing region 202A, 202B, 202C, 202D and 202E may be treated to mimic stain characteristics (e.g., stain density or color) of one or more particular interferent gaseous substances associated with the respective testing region 202A, 202B, 202C, 202D and 202E.

While FIG. 2A and FIG. 2B provide examples of gas-sensitive substrates 200A and 200B, it is noted that the scope of the present disclosure is not limited to the examples shown in FIG. 2A and FIG. 2B. In some examples, an example gas detecting apparatus may comprise one or more additional and/or alternative elements, and/or may be structured/positioned differently than those illustrated in FIG. 2A and FIG. 2B. For example, the testing region(s) of a gas-sensitive substrate in accordance with the present disclosure may comprise squares, triangles or non-geometric shapes and may be arranged differently from the example provided in FIG. 2B.

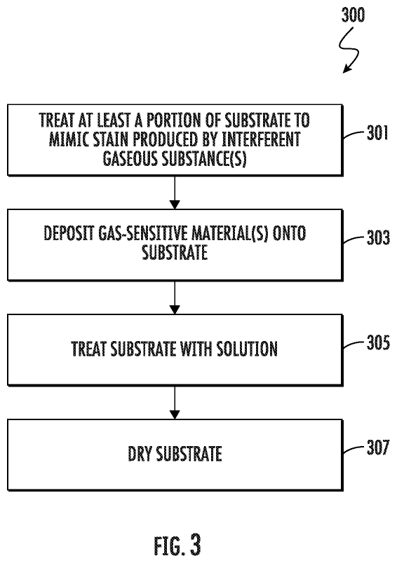

Referring now to FIG. 3, a flowchart diagram depicting an example method 300 for producing a gas-sensitive substrate in accordance with various embodiments of the present disclosure is provided.

Beginning at step/operation 301, the method may begin with treating at least a portion (e.g., surface) of a substrate (e.g., a paper-based and/or plastic-based media) to mimic stain characteristics (e.g., stain density or color) produced by at least one interferent gaseous substance. For example, at least a portion of the substrate may be dyed using a non-reactive pigment such that a base color of the substrate is similar to that of an example interferent gaseous substance. By way of example, Nitrogen Dioxide and Ozone belong to a family of gaseous substances (i.e., Oxidizers). A chemically treated substrate may produce a yellow stain in response to making contact with Ozone. In one example, in order to produce a gas-sensitive substrate for detecting and/or measuring a concentration of Nitrogen Dioxide that will not react to Ozone, at least a portion of the example substrate may be dyed yellow. In another example, Fluoride and Chlorine also belong to a family of gaseous substances (i.e., Oxidizers). A chemically treated substrate may produce a grey stain in response to making contact with Chlorine. Accordingly, in order to produce a gas-sensitive substrate for detecting and/or measuring a concentration of Fluoride that will not react to Chlorine, the example substrate may be dyed a color or otherwise treated so as to appear similar to a stain produced by Chlorine.

The substrate may be treated (e.g., dyed with non-reactive pigment(s)) in a variety of ways, such as by using sprayers, drop-depositing devices, or pumps. In particular, an example treatment unit may include micro-solenoid valves, inkjet printers, or piezoelectric, acoustic, or thermal valves. An example sprayer may include aerosol or spray-based dispensing heads. In some examples, a liquid pump may be used to deposit non-reactive pigments(s). An example pump may comprise orifices or tubes from which small volumes of gas-sensitive material(s) are pumped. An applied force (such as peristaltic, syringe, or capillary forces) may be used to deposit the non-reactive pigments. In some examples, the entire example substrate or a portion of the example substrate may be submerged in a container (e.g., vat, tub or the like) of an example non-reactive pigment.

Subsequent to step/operation 301, the method 300 proceeds to step/operation 303. At step/operation 303, subsequent to treating at least a portion of the substrate to mimic stain characteristics (e.g., stain density or color) produced by at least one interferent gaseous substance, gas-sensitive material(s) may be deposited upon at least a portion of the example substrate. The gas-sensitive material(s) may be or comprise one or more chemical reagents or the like. In various examples, a particular substrate may be configured based on a particular reaction principle. For example, a reaction principle for an Oxidizer may be Oxidation of Aromatic Amine.

In various examples, the gas-sensitive material(s) may be deposited directly onto treated/dyed portions of the example substrate. Additionally and/or alternatively, the gas-sensitive material(s) may be deposited onto untreated regions of the substrate. In some examples, depositing gas-sensitive material(s) onto at least a portion the substrate may comprise determining various properties of an example substrate (e.g., a moisture content of the example substrate) and/or environmental conditions in order to determine one or more parameters (e.g., humidity, temperature and/or the like) for depositing the gas-sensitive material(s). By way of the example, the substrate may pass through one or more controlled chambers while the gas sensitive material(s) are deposited thereon. In some examples, environmental conditions within the one or more controlled chambers (e.g., humidity, temperature and/or the like) may be controlled to bring the moisture content or other property of the substrate to a specific value or within a specific range. In some examples, the tension of the substrate may be modified based on the moisture content or other property of the substrate.

In some examples, a dispensing unit may be utilized to deposit gas-sensitive material(s) onto at least a portion (e.g., surface, region(s) and/or the like) of the example substrate. In one example, the example dispensing unit may extract the substrate, such as from a roll, and move it in a direction past a deposition location. At the deposition location, one or more gas-sensitive materials can be applied to/selectively deposited onto the substrate. The gas-sensitive material(s) may be deposited in a variety of ways, such as by using sprayers, drop-depositing devices, or pumps. In particular, an example dispensing unit may include micro-solenoid valves, inkjet printers, or piezoelectric, acoustic, or thermal valves. An example sprayer may include aerosol or spray-based dispensing heads. In some examples, a liquid pump may be used to deposit the gas-sensitive material(s). An example pump may comprise orifices or tubes from which small volumes of gas-sensitive material(s) are pumped. An applied force (such as peristaltic, syringe, or capillary forces) may be used to deposit the gas-sensitive material(s).

Subsequent to step/operation 303, the method 300 proceeds to step/operation 305. At step/operation 305, subsequent to depositing gas-sensitive material(s) onto at least a portion (e.g., surface, regions and/or the like) of the example substrate, the substrate is treated with a solution. In various examples, the solution may be an alcohol-based solution, a solvent-based solution or the like. By way of example, the example substrate may be at least partially submerged in a container of example solution. The example solution may be applied to the substrate using other techniques including any of the techniques described above in relation to depositing the gas-sensitive material(s). In one example, the example solution may be sprayed directly onto gas-sensitive region(s).

Subsequent to step/operation 305, the method 300 proceeds to step/operation 307. At step/operation 307, subsequent to treating the substrate with a solution, the substrate is dried. In various examples, the substrate may be dried in a controlled environment at a particular humidity, temperature and air composition (e.g., an oven, chamber or the like). In some examples, parameters of the controlled environment may be adjusted over time. In some examples, characteristics of the substrate may be measured in order to adjust parameters of the controlled environment. This could include, for example, measuring the moisture content of the substrate using O—H stretch or other suitable technique. One or more characteristics of the controlled environment (e.g., oven or chamber) may be adjusted. For example, the temperature, humidity, or composition of air in the controlled chamber may be adjusted in order to bring the moisture content or other property of the substrate to a specific value or within a specific range. Tension of the example substrate any other or additional action(s) may be taken based on the moisture content or other property of the substrate. In some examples, a controller may be utilized to automatically/dynamically measure and adjust system characteristics based on temperature, humidity, or other measurements. Additionally, the speed at which the substrate may be modified (e.g., increased or decreased) in order to produce a substrate with particular properties. In some examples, the example substrate may be dried in a temperature below 100 C or at a boiling point of an example solvent.

Although FIG. 3 illustrates one example of a method 300 for producing a gas-sensitive substrate, other methods may be utilized. For example, while shown as a series of operations/steps, various operations/steps in FIG. 3 could overlap, occur in parallel, occur in a different order, or occur multiple times.

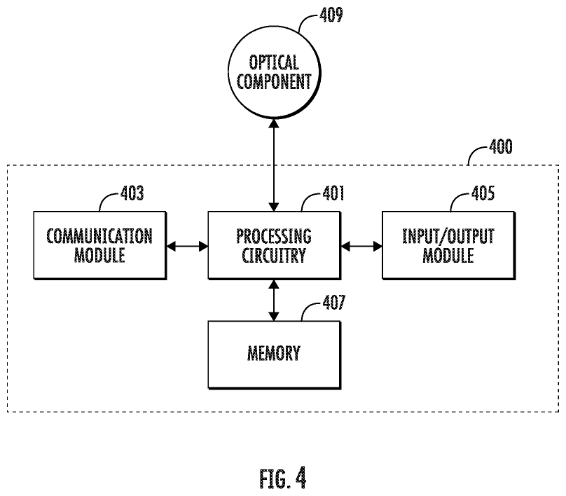

Referring now to FIG. 4, a schematic diagram depicting an example controller component 400 of an example gas detecting apparatus in electronic communication with various other components in accordance with various embodiments of the present disclosure. As shown, the controller component 400 comprises processing circuitry 401, a communication module 403, input/output module 405, a memory 407 and/or other components configured to perform various operations, procedures, functions or the like described herein.

As shown, the controller component 400 (such as the processing circuitry 401, communication module 403, input/output module 405 and memory 407) is electrically coupled to and/or in electronic communication with an optical component 409 (e.g., comprising a gas detecting element). The optical component 409 may be similar to the optical component 105 described above in connection with FIG. 1. As depicted, the optical component 409 may exchange (e.g., transmit and receive) data with the processing circuitry 401 of the controller component 400.

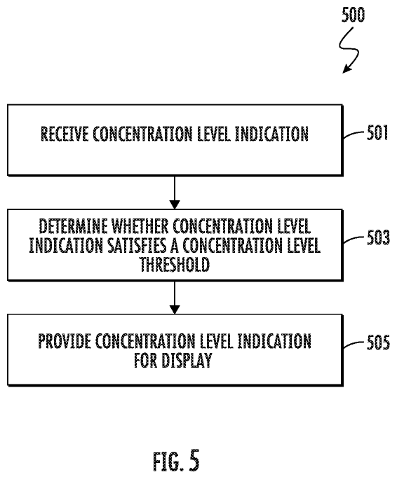

The processing circuitry 401 may be implemented as, for example, various devices comprising one or a plurality of microprocessors with accompanying digital signal processors; one or a plurality of processors without accompanying digital signal processors; one or a plurality of coprocessors; one or a plurality of multi-core processors; one or a plurality of controllers; processing circuits; one or a plurality of computers; and various other processing elements (including integrated circuits, such as ASICs or FPGAs, or a certain combination thereof). In some embodiments, the processing circuitry 401 may comprise one or more processors. In one exemplary embodiment, the processing circuitry 401 is configured to execute instructions stored in the memory 407 or otherwise accessible by the processing circuitry 401. When executed by the processing circuitry 401, these instructions may enable the controller component 400 to execute one or a plurality of the functions as described herein. No matter whether it is configured by hardware, firmware/software methods, or a combination thereof, the processing circuitry 401 may comprise entities capable of executing operations according to the embodiments of the present invention when correspondingly configured. Therefore, for example, when the processing circuitry 401 is implemented as an ASIC, an FPGA, or the like, the processing circuitry 401 may comprise specially configured hardware for implementing one or a plurality of operations described herein. Alternatively, as another example, when the processing circuitry 401 is implemented as an actuator of instructions (such as those that may be stored in the memory 407), the instructions may specifically configure the processing circuitry 401 to execute one or a plurality of algorithms and operations described herein, such as those discussed with reference to FIG. 5.

The memory 407 may comprise, for example, a volatile memory, a non-volatile memory, or a certain combination thereof. Although illustrated as a single memory in FIG. 4, the memory 407 may comprise a plurality of memory components. In various embodiments, the memory 407 may comprise, for example, a hard disk drive, a random access memory, a cache memory, a flash memory, a Compact Disc Read-Only Memory (CD-ROM), a Digital Versatile Disk Read-Only Memory (DVD-ROM), an optical disk, a circuit configured to store information, or a certain combination thereof. The memory 407 may be configured to store information, data, application programs, instructions, and etc., so that the controller component 400 can execute various functions according to the embodiments of the present disclosure. For example, in at least some embodiments, the memory 407 is configured to cache input data for processing by the processing circuitry 401. Additionally or alternatively, in at least some embodiments, the memory 407 is configured to store program instructions for execution by the processing circuitry 401. The memory 407 may store information in the form of static and/or dynamic information. When the functions are executed, the stored information may be stored and/or used by the controller component 400.

The communication module 403 may be implemented as any apparatus included in a circuit, hardware, a computer program product or a combination thereof, which is configured to receive and/or transmit data from/to another component or apparatus. The computer program product comprises computer-readable program instructions stored on a computer-readable medium (for example, the memory 407) and executed by a controller component 400 (for example, the processing circuitry 401). In some embodiments, the communication module 403 (as with other components discussed herein) may be at least partially implemented as the processing circuitry 401 or otherwise controlled by the processing circuitry 401. In this regard, the communication module 403 may communicate with the processing circuitry 401, for example, through a bus. The communication module 403 may comprise, for example, antennas, transmitters, receivers, transceivers, network interface cards and/or supporting hardware and/or firmware/software, and is used for establishing communication with another apparatus. The communication module 403 may be configured to receive and/or transmit any data that may be stored by the memory 407 by using any protocol that can be used for communication between apparatuses. The communication module 403 may additionally or alternatively communicate with the memory 407, the input/output module 405 and/or any other component of the controller component 400, for example, through a bus.

In some embodiments, the controller component 400 may comprise an input/output module 405. The input/output module 405 may communicate with the processing circuitry 401 to receive instructions input by the user and/or to provide audible, visual, mechanical or other outputs to the user. Therefore, the input/output module 405 may comprise supporting devices, such as a keyboard, a mouse, a display, a touch screen display, and/or other input/output mechanisms. Alternatively, at least some aspects of the input/output module 405 may be implemented on a device used by the user to communicate with the controller component 400. The input/output module 405 may communicate with the memory 407, the communication module 403 and/or any other component, for example, through a bus. One or a plurality of input/output modules and/or other components may be included in the controller component 400.

For example, the optical component 409 may be similar to optical component 105 described above with regard to FIG. 1. For example, optical component 409 may generate measurements indicating a concentration level of a target gaseous substance in a sample gaseous substance disposed within a gas flow channel of a gas detecting apparatus and transmit a concentration level indication to the processing circuitry 401.

Referring now to FIG. 5, a flowchart diagram illustrating an example method 500/operations in accordance with various embodiments of the present disclosure is provided.

In some examples, the method 500 may be performed by a processing circuitry (for example, but not limited to, an application-specific integrated circuit (ASIC), a central processing unit (CPU)). In some examples, the processing circuitry may be electrically coupled to and/or in electronic communication with other circuitries of the example apparatus, such as, but not limited to, a humidity sensing component, a dehumidifier component, a gas detecting, a memory (such as, for example, random access memory (RAM) for storing computer program instructions), and/or a display circuitry (for rendering readings on a display).

In some examples, one or more of the procedures described in FIG. 5 may be embodied by computer program instructions, which may be stored by a memory (such as a non-transitory memory) of a system employing an embodiment of the present disclosure and executed by a processing circuitry (such as a processor) of the system. These computer program instructions may direct the system to function in a particular manner, such that the instructions stored in the memory circuitry produce an article of manufacture, the execution of which implements the function specified in the flow diagram step/operation(s). Further, the system may comprise one or more other circuitries. Various circuitries of the system may be electronically coupled between and/or among each other to transmit and/or receive energy, data and/or information.

In some examples, embodiments may take the form of a computer program product on a non-transitory computer-readable storage medium storing computer-readable program instruction (e.g., computer software). Any suitable computer-readable storage medium may be utilized, including non-transitory hard disks, CD-ROMs, flash memory, optical storage devices, or magnetic storage devices.

The example method 500 begins at step/operation 501. At step/operation 501, a processing circuitry (such as, but not limited to, the processing circuitry 401 of the controller component 400 illustrated in connection with FIG. 4, discussed above) receives a concentration level indication associated with a target gaseous substance in a sample. In some embodiments, an optical component (such as, but not limited to, the optical component 409 illustrated in connection with FIG. 4) may transmit a concentration level indication associated with a target gaseous substance to the processing circuitry. The concentration level indication may be generated in response to a gas-sensitive substrate making contact with at least one target gaseous substance disposed within a gas flow channel of a gas detecting apparatus such that a chemical reaction that produces a stain with particular characteristics occurs. In various examples, concentration of the target gaseous substance(s) may be measured in parts-per-million (ppm), parts-per-billion (ppb), milligrams-per-cubic-meter (mg/m3), or the like. In some examples, the example optical component may periodically provide a concentration level indication. In some examples, the optical component may provide a concentration level indication in response to a request (e.g., in response to receiving a control signal or indication from the processing circuitry).

Subsequent to step/operation 501, the example method 500 proceeds to step/operation 503. At step/operation 503, the processing circuitry determines whether the concentration level indication satisfies a concentration level threshold.

As noted above, in some embodiments, the concentration level threshold may be a measure of concentration associated with a stain resulting from a target gaseous substance making contact with a gas-sensitive substrate (e.g., a stain density). The concentration level threshold may be a predetermined and/or configurable threshold. In some embodiments, an operator of the gas detecting apparatus may select the concentration level threshold. In one example, a concentration level threshold associated with a gas-sensitive substrate (e.g., gas-sensitive region or testing region) configured to detect and/or measure a concentration of Fluorine may be 1 ppm. In another example, a concentration level threshold associated with a gas-sensitive substrate (e.g., gas-sensitive region or testing region) configured to detect and/or measure a concentration of Chlorine Dioxide may be 100 ppb.

In some embodiments, the processing circuitry may determine that the concentration level indication does not satisfy a concentration level threshold if the concentration level indicated by the concentration level indication is equal to or below the concentration level threshold. In some embodiments, the processing circuitry may determine that the concentration level indication satisfies a concentration level threshold if the concentration level indicated by the concentration level indication is above the concentration level threshold.

Subsequent to step/operation 503, the method 500 proceeds to step/operation 505. At step/operation 505, if the processing circuitry determines that the concentration level threshold is satisfied, the processing circuitry may provide a concentration level indication and/or warning indication for display. For example, the processing circuitry may provide or generate a concentration level indication, warning indication, alert, and/or the like for presentation. In some examples, the concentration level indication and/or warning indication may be provided for display via a display or user interface of an example gas detecting apparatus. Additionally and/or alternatively, the concentration level indication and/or warning indication may be provided for display via another user computing device in electronic communication with the example gas detecting apparatus. Accordingly, using the techniques described herein, the accuracy of measurements and corresponding warning indications/alerts provided by a gas detecting apparatus may be improved while preventing false alarms by interferent gaseous substances.

Many modifications and other embodiments of the present disclosure set forth herein will come to mind to one skilled in the art to which these embodiments pertain having the benefit of the teachings presented in the foregoing descriptions and the associated drawings. Therefore, it is to be understood that the disclosure are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A gas detecting apparatus comprising:
a gas-sensitive substrate at least partially disposed within a gas flow channel of the gas detecting apparatus, wherein:
at least a portion of the gas-sensitive substrate is treated to mimic stain characteristics produced by at least one interferent gaseous substance, and
the gas-sensitive substrate is configured to produce a stain in response to making contact with at least one target gaseous substance disposed within the gas flow channel.

2. The gas detecting apparatus of claim 1, further comprising:
an optical component configured to generate a concentration level indication associated with the at least one target gaseous substance in the gas flow channel.

3. The gas detecting apparatus of claim 2, wherein the optical component comprises a gas detecting element.

4. The gas detecting apparatus of claim 2, further comprising a controller component in electronic communication with the gas detecting element, the controller component configured to:
receive the concentration level indication from the optical component;
determine whether the concentration level indication satisfies a concentration level threshold; and
in response to determining that the concentration level indication satisfies the concentration level threshold, generate a warning indication.

5. The gas detecting apparatus of claim 1, wherein the at least one target gaseous substance comprises an Oxidizer.

6. The gas detecting apparatus of claim 5, wherein the at least one target gaseous substance comprises one or more of Chlorine, Fluorine, Nitrogen Dioxide and Chlorine Dioxide.

7. The gas detecting apparatus of claim 5, wherein a color of the gas-sensitive substrate is yellow.

8. The gas detecting apparatus of claim 5, wherein a color of the gas-sensitive substrate is grey.

9. The gas detecting apparatus of claim 5, wherein the at least one interferent gaseous substance comprises one or more of Ozone, Sulphur Dioxide and Hydrogen Peroxide.

10. The gas detecting apparatus of claim 1, wherein the gas-sensitive substrate is treated with an alcohol-based or solvent-based solution.

11. The gas detecting apparatus of claim 1, wherein the gas-sensitive substrate is heated or dried.

12. A method for manufacturing a gas-sensitive substrate configured to produce a stain in response to making contact with at least one target gaseous substance, the method comprising:
treating at least a portion of a substrate to mimic stain characteristics produced by at least one interferent gas; and
depositing at least one gas-sensitive material unto the substrate.

13. The method of claim 12, wherein the at least one target gaseous substance comprises an Oxidizer.

14. The method of claim 13, wherein the at least one target gaseous substance comprises one or more of Chlorine, Fluorine, Nitrogen Dioxide and Chlorine Dioxide.

15. The method of claim 13, wherein a color of the gas-sensitive substrate is yellow.

16. The method of claim 13, wherein a color of the gas-sensitive substrate is grey.

17. The method of claim 13, wherein the at least one interferent gaseous substance comprises one or more of Ozone, Sulphur Dioxide and Hydrogen Peroxide.

18. The method of claim 12, wherein the gas-sensitive substrate comprises a paper tape.

19. The method of claim 12, further comprising:
treating the substrate with an alcohol-based or solvent-based solution.

20. The method of claim 12, further comprising heating or drying the substrate.
